(19) United States
(12) Patent Application Publication
(10) Pub. No.: US 2024/0325327 A1
(43) Pub. Date: Oct. 3, 2024

(54) METHOD FOR ATTENUATING NEUROINFLAMMATION, AMYLOIDOPATHY AND TAUOPATHY

(71) Applicant: Trustees of Dartmouth College, Hanover, NH (US)

(72) Inventors: Ta Yuan CHANG, Etna, NH (US); Catherine C.Y. CHANG, Etna, NH (US); Adrianna DE LA TORRE, Lyme Somerville, NH (US); HaiBo LI, Hanover, NH (US); Thao N. HUYNH, West Lebanon, NH (US); Junghoon LEE, West Lebanon, NH (US)

(21) Appl. No.: 18/599,832

(22) Filed: Mar. 8, 2024

Related U.S. Application Data
(60) Provisional application No. 63/454,774, filed on Mar. 27, 2023.

(51) Int. Cl. A61K 31/167; A61K 9/127; A61K 9/51; A61K 45/06; A61P 25/00

(52) U.S. Cl. CPC A61K 31/167; A61K 9/1271; A61K 9/5123; A61K 45/06; A61P 25/00

(57) ABSTRACT

The present invention provides a method for preventing or treating neuroinflammation, amyloidopathy or tauopathy by inhibiting Acyl-CoA:Cholesterol Acyltransferase activity with a brain-permeable inhibitor encapsulated in a stealth liposome-based nanoparticle.

### INTRODUCTION

This application claims the benefit of priority from U.S. Provisional Application Ser. No. 63/454,774 filed Mar. 27, 2023, the teachings of which are herein incorporated by reference in their entirety.

This invention was made with government support under Grant Number AG063544 awarded by the National Institutes of Health. The government has certain rights in the invention.

### BACKGROUND

Chronic neuroinflammation is a hallmark of late onset Alzheimer's disease (LOAD) and in many other neurodegenerative diseases, as well as in aging. In the central nervous system (CNS), the Toll-Like Receptor 4 (TLR4) is expressed in microglia, astrocytes, oligodendrocytes, and neurons. It is a transmembrane receptor protein that recognizes diverse pathogen-derived ligands including lipopolysaccharides (LPS), and various tissue damage-related ligands including oligomeric amyloid peptide fragment Aβ_{1_42}, heat-shock proteins (especially HSP60 and HSP70), high mobility group box 1 (HMGB1), hyaluronic acid, fibronectin, galectin-3, and the like. TLR4 is a LOAD susceptibility gene. In LOAD, aging, and several neurodegenerative diseases, TLR4 plays a key role in mediating pro-inflammatory responses, such as pro-inflammatory cytokine production in the CNS, when bound to various ligands including LPS.

Cholesterol is stored as cholesteryl esters. For cholesterol esterification, there are two Acyl-CoA:Cholesterol Acyltransferase (ACAT) genes, SOAT1 and SOAT2, encoding two homologous but distinct enzymes, ACAT1 and ACAT2. Both enzymes use long-chain fatty acyl-CoAs and sterols with 3-beta-OH, including cholesterol and various oxysterols as their substrates. ACAT1 is the major cholesterol storage enzyme in the brain. Both compound K-604 (Ki=0.5 μM) and compound F12511 (Ki=0.04 μM) are high-affinity, ACAT1-specific, small-molecule inhibitors. Both inhibitors had passed phase I clinical safety tests for treating cardiovascular disease. K-604 is rather hydrophilic, while F12511 is extremely hydrophobic and both inhibitors tightly bind to ACAT1. F12511 preferentially inhibits ACAT1 but it also inhibits ACAT2 (Ki=0.11 μM).

Using a custom-made slow-release pellet method, the isoform-non-specific ACAT inhibitor CP113,818 has been shown to reduce amyloidopathy and rescue cognitive deficits in a mouse model for Alzheimer's Disease (AD) (Hutter-Paier, et al. (2004) Neuron 44(2):227-38). Similarly, the isoform-non-specific ACAT inhibitor, CI 1011, has been shown to reduce amyloidopathy in a mouse model for AD. In this respect, a method for preparing a water-soluble formulation containing CI 1011 has been described, wherein CI 1011 is incorporated with albumin such that CI 1011 can achieve relatively high concentration in the blood (Lee, et al. (2015) ACS Nano 9(3):2420-32). However, it has not been shown that CI 1011 is able to enter the brain using such a formulation.

### SUMMARY OF THE INVENTION

The present invention provides a method for reducing or attenuating neuroinflammation, amyloidopathy or tauopathy by administering to a subject in need thereof a pharmaceutical composition including nanoparticles composed of an outer lipid envelope and a core, the outer lipid envelope including distearoyl-sn-glycero-3-phosphoethanolamine-poly(ethylene glycol) and phosphatidylcholine, the core including an Acyl-CoA:Cholesterol Acyltransferase (ACAT) inhibitor having the structure:

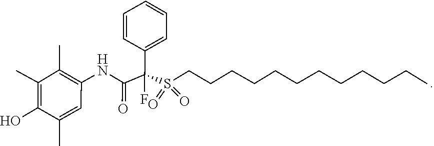 [SMILES: CCCCCCCCCCCCS(=O)(=O)[C@](F)(C(=O)Nc1cc(C)c(O)c(C)c1C)c1ccccc1]

### BRIEF DESCRIPTION OF THE DRAWINGS

FIG. 1 shows that F26 is a potent ACAT1 inhibitor. F26 synthesized and purified to 98% and tested for ACAT1 inhibition. Its EC₅₀ was 5.8 nM which is almost 7 times more potent than F12511.

FIG. 2A-2B show ACAT inhibition in forebrain, cerebellum, liver, adrenals over time after a single IV injection of nanoparticle-formulated F12511 or nanoparticle-formulated F26. FIG. 2A shows ACAT1 levels achieved after exposure to nanoparticle-formulated F26 (at low and high doses as indicated) vs. nanoparticle-formulated F12511 (at high dose only). After delivering by IV for 6 hours, various tissues (i.e., forebrain, cerebellum/brainstem, adrenal gland and liver) were isolated and subjected to ACAT1 inhibition measurement. FIG. 2B, Efficacy of ACAT inhibition after IV injection in mice of nanoparticle-formulated F12511 vs. F26. Four hours after IV injection, animals were sacrificed and various tissues (i.e., forebrain, cerebellum/brainstem, adrenal gland and liver) were isolated and subjected to ACAT1 inhibition measurement. N=2-3 WT mice/group. N=1 A1^{-/-} mouse. Statistical analysis, 2-way ANOVA. *P<0.05; **P<0.01; ***P<0.001.

FIG. 3 shows the time course of nanoparticle F26's effect on ACAT activity in vivo. Treatment groups were divided into the following four groups: (1) PBS; (2) vehicle (DSPE-PEG/PC) group; (3) nanoparticle F26 treatment for 24-hour group; and (4) nanoparticle F26 treatment for 48-hour group. Wild-type (C57BL/6) mice were retro-orbitally treated with 12 mM of nanoparticle F26 for either 24 or 48 hours, and tissues were collected to measure ACAT enzyme activity. Significant differences were observed in the nanoparticle F26 treatment groups compared with the PBS and vehicle group (*p<0.05, **p<0.01, ****p<0.0001). n=2-6 mice/group.

FIG. 4A-4D show that myeloid Acat1 inactivation suppresses expression of certain pro-inflammatory genes while inducing expression of certain anti-inflammatory genes after LPS injection to mice. Two-month-old Acat1^{flox/flox} or Acat1^{flox/flox} LyzM^{Cre} (myeloid Acat1 knockout) mice were treated with single peritoneal injections of LPS at 5 mg/kg body weight. 24 hours later, mice were sacrificed, mRNA was extracted from hippocampus (FIG. 4A and FIG. 4B) and cortex (FIG. 4C and FIG. 4D) for qPCR to monitor expression of various pro-inflammatory genes (FIG. 4A and FIG. 4C) or anti-inflammatory genes (FIG. 4B and FIG. 4D). * P<0.05; ** P<0.01; *** P<0.001.

FIG. 5A-5C show that inhibiting ACAT1 in N9 microglial cells attenuates inflammatory responses to LPS. Cells were seeded at 1×10⁵/well onto 12-well plates in RPMI-1640 with 10% bovine calf serum. FIG. 5A and FIG. 5B, Cells were treated with DMSO (control) or DMSO with 0.5 μM Acat1 inhibitor K-604 for 4 hours, then treated without or with 10, 100, or 1000 ng/mL LPS. Six hours later, mRNA was extracted, and qPCR was performed to monitor pro-inflammatory (FIG. 5A) or anti-inflammatory (FIG. 5B) gene expression. FIG. 5C, N9 cells were treated with DMSO (control) or DMSO with 0.5 μM K-604 for 4 hours then treated without or with 100 ng/mL LPS. Thirty minutes later, proteins were extracted for western blot analysis for total IκB-α, and for phosphorylated IκB-α (p-IκB-α). *P<0.05; ** P<0.01.

FIG. 6 shows that inhibiting ACAT1 in microglial N9 cells increases expression of the receptor IL-4Rα (the key receptor for the anti-inflammatory cytokine IL4). Microglial N9 cells grown in RPMI including 10% bovine calf serum were treated with DMSO without or with the ACAT1 inhibitor K-604 at 0.5 μM for 4 or 12 hours as indicated. Cells were harvested for immunoblot analysis for IL-4Rα. Vinculin was the loading control. Data expressed as mean+/−SEM, * p<0.05, ** p<0.01.

FIG. 7A-7B show that ACAT1 blockade upregulates IL-4Rα expression in mouse myelin debris-treated N9 (FIG. 7A) and HMC3 (FIG. 7B) microglia. Cells were grown until 90% confluency and subsequently treated with DMSO without or with K-604, or with myelin debris (purified as described by Rolfe, et al. (2017) J. Vis. Exp. 130:56322) for 21 hours. IL-4Rα expression was determined by convention methods. 25 μg/mL cholesterol equals 38 μg/mL total myelin debris protein. Data are expressed as mean±SEM. *P<0.05; **P<0.01.

FIG. 8A-8B show that IL-4Rα protein content increases in human myelin debris-treated HMC3 cells after 48 hours co-incubation with either K-604 (FIG. 8A) or F12511 (FIG. 8B). Cells were grown until 90% confluency and subsequently treated with DMSO without or with ACAT inhibitor, with or without myelin debris for 21 hours. Protein was isolated and analyzed by western blot analysis. Vinculin was the loading control. Values were calculated based on the value with no myelin and K-604 or F12511 treatment as 1. Data are expressed as mean±SEM. *P<0.05; **P<0.01.

FIG. 9A-9B show that ABCA1 protein content increases in human myelin debris-treated HMC3 cells after 24 hours co-incubation with either K-604 or F12511 as the ACAT inhibitor. HMC3 cells were grown in MEM plus 10% BCS, with 0.1% DMSO as the control, or 0.5 μM K-604 (FIG. 9A) or 0.5 μM F12511 incubated with or without human myelin debris at 25 μg/mL cholesterol for 24 hours (FIG. 9B). Afterward, cells were harvested for western blot analyses. Lysates of HEK293 cells and lysates of HMC3 cells treated with 10 μM LXR agonist T0901317 for 24 hours were respectively used as negative and positive controls for ABCA1 signals. Values were based on the value of lysates from cells with no myelin or K-604 or F12511 treatments as 1. Data are expressed as mean±SEM.

FIG. 10A-10B show that nanoparticle and nanoparticle F synergistically reduce TLR4 protein content in ApoE3 (FIG. 10A) and ApoE4 (FIG. 10B) female mouse forebrains (16-20-month-old mice). ApoE3 and ApoE4 female mice were injected through IV or retro-orbital route (alternate injection route per day) for 14 days. Afterward, mice were perfused with 20 mL of cold PBS, and brains were collected. Forebrain tissues were flash frozen on dry ice and stored at −80° C. until analysis. Tissues were then minced and homogenized in sucrose homogenization buffer. Western blot images were captured and quantified using Li-Cor Image Studio software. Unpaired student t-test (two-tailed) were performed, *P<0.05, N=3-5 mice/group. NP: nanoparticle. F stands for F12511.

FIG. 11A-11E show that nanoparticle and nanoparticle F alter pro-inflammatory cytokines profile in female ApoE3 and ApoE4 (16-20 month; after 2-wk daily IV/RO injections. Mice were perfused with 20 mL of cold 1×PBS, brains were collected and homogenized. Whole brain homogenates were analyzed using MILLIPLEX MAP Mouse Cytokine/Chemokine Magnetic Bead Panel (32-plex) and data were normalized using total protein content determined by Lowry assay. (FIG. 11A) IL-1a, (FIG. 11B) IL-1b, (FIG. 11C) TNF-α, (FIG. 11D) IL-12p40, and (FIG. 11E) IL-12p70 cytokines were plotted individually with ApoE3 or ApoE4 forebrain cytokines content. Values obtained from each individual cytokine from PBS injected animal was normalized to 1. Unpaired student t-test (two-tailed) were performed, *P<0.05, n=3-5 mice/group. PBS: phosphate-buffered saline. NP: nanoparticle. NPF: F12511 nanoparticle.

### DETAILED DESCRIPTION OF THE INVENTION

This invention employs stealth nanoparticles encapsulating an Acyl-CoA:Cholesterol Acyltransferase (ACAT) inhibitor for reducing or attenuating neuroinflammation, amyloidopathy and/or tauopathy. Stealth nanoparticles composed of distearoyl-sn-glycero-3-phosphoethanolamine-poly(ethylene glycol) and phosphatidylcholine, when delivered by intravenous injection or by intraperitoneal injection at a concentration of 30 mM, have been shown to provide a concentration of ACAT inhibitor in the blood of a subject that exceeds 20 μM for at least 4 hours, thereby effectively allowing for delivery of the ACAT inhibitor into brain cells. A 2-week intravenous delivery of nanoparticle encapsulated ACAT inhibitor to aging 3×Tg AD mice ameliorated amyloidopathy and reduced hyperphosphorylated Tau and non-phosphorylated Tau. Nanoparticle encapsulated ACAT inhibitor ameliorated neuroinflammation in aging ApoE3/E4 knock-in mice. ACAT1 inhibition also attenuates LPS-mediated inflammatory responses by diminishing the TLR4-mediated pro-inflammatory signaling cascade and inhibits cholesterol esterification in neuronal and microglial cell lines, without being toxic to said cells. Moreover, the incorporation of at least 6 mM phosphatidylcholine in the nanoparticle envelope provides for encapsulation of high concentrations of hydrophobic compounds. Further, a significant portion of the stealth nanoparticle, by itself, can permeate the blood-brain carrier, and enters brain tissue, ACAT inhibitor encapsulated nanoparticles can reduce hyperphosphorylated and aggregated htau and reduce amyloid burden. Accordingly, the present invention provides methods for reducing or attenuating neuroinflammation, amyloidopathy and/or tauopathy resulting from pro-inflammatory responses in the CNS and preventing or treating LOAD as well as other related neurodegenerative diseases such as vascular dementia, tauopathy, Parkinson's disease, frontotemporal dementia, amyotrophic lateral sclerosis, and adult glioma by administering encapsulated stealth nanoparticles that target and inhibit ACAT1 in brain tissue.

A nanoparticle of this invention denotes a sphere with a mean diameter of less than 300 nm, which has a circular, unilamellar lipid wall or envelope and a central component or core that contains an analide derivative (see US 2006/0135785 A1) and referred to herein as F26, and optionally one or more additional therapeutic ingredients. Nanoparticles useful in the method of this invention are “stealth” in that they are not detected and sequestered and/or degraded, or they are detected and then sequestered and/or degraded at very low levels, and/or they are detected and then sequestered and/or degraded by the immune system of the host to which they are administered only after significant exposure times in the brain. In this respect, the nanoparticles of the present invention exhibit long biological circulating times, having a a half-life in the blood compartment of greater than 2, 4, 6, 8, 10, 12, or 24 hours, making it possible for a significant portion of the nanoparticles administered (approximately 0.5% (Saucier-Sawyer et al. (2015) J. Drug Target. 23(7-8):736-49) to enter the brain. Once in the brain, a nanoparticle composed of DSPE-PEG and PC can exert various biological effects on various cell types in the CNS (e.g., reduce hyperphosphorylated and aggregated htau and reduce amyloid burden). Further, the nanoparticles can carry a relatively high content of active ingredient through a subject thereby providing reduced toxicity compared with non-encapsulated drug.

In certain aspects, the nanoparticles have at least one dimension in the range of about 10 nm to about 300 nm, including any integer value between 1 nm and 300 nm (including about 20, 30, 40, 50, 60, 70, 80, 90, 100, 200, 250, and 300). In certain aspects, the nanoparticles have at least one dimension that is about 15 nm to 250 nm. Particle size can be determined using any method known in the art, including, but not limited to, sedimentation field flow fractionation, photon correlation spectroscopy, disk centrifugation, dynamic light scattering, and electron microscopy.

The stealth nanoparticles are composed of a core which is liquid or semi-liquid at ambient temperature, and a lipid envelope composed of phosphatidylcholine (PC) and a biodegradable phospholipid, preferably a pegylated phospholipid, most preferably distearoyl-sn-glycero-3-phosphoethanolamine-poly(ethylene glycol)ₓ (DSPE-PEGₓ), in which x represents the size of the PEG molecule in g/mol. In some aspects, the molar mass of the PEG component is in the range of about 100 to about 20,000 g/mol, including about 500 g/mol, about 600 g/mol, about 700 g/mol, about 800 g/mol, about 900 g/mol, about 1000 g/mol, about 2000 g/mol, about 3000 g/mol, about 5000 g/mol, about 10,000 g/mol, about 15,000 g/mol, and about 20,000 g/mol. In certain aspects, the molar mass of the PEG component is greater than or equal to 1000 g/mol, preferably greater than or equal to 2000 g/mol. In some aspects, DSPE-PEG₁₀₀₀, DSPE-PEG₂₀₀, DSPE-PEG₃₀₀₀ or DSPE-PEG₅₀₀₀ is used. In particular aspects, DSPE-PEG₂₀₀₀ or DSPE-PEG₅₀₀₀ is used. Preferably, the nanoparticles include between 20 mM and 40 mM, preferably between 25 mM and 35 mM, most preferably 30 mM of the pegylated phospholipid (e.g., DSPE-PEG₂₀₀₀ or DSPE-PEG₅₀₀₀).

Ideally, the phosphatidylcholine used in the preparation of the nanoparticles is purified to at least 95%, 96%, 97%, 98%, 99% or 100% homogeneity. In this respect, the phosphatidylcholine has less than 5%, 4%, 3%, 2%, 1%, 0.5%, or 0.1% impurities such as other phosphatides (e.g., phosphatidylethanolamine, phosphatidylserine, phosphatidic acid, and phosphatidylinositol), triglycerides, fatty acids, or carbohydrates. In particular aspects, the nanoparticles include between 2 mM and 10 mM, preferably between 4 mM and 8 mM, most preferably 6 mM phosphatidylcholine.

The term “brain-permeable” refers to the ability of a drug to permeate or cross the blood brain barrier. In some aspects, animal pharmacokinetic (pK) studies, such as mouse pharmacokinetic/blood-brain barrier studies, can be used to determine or predict brain permeability. For example, various concentrations of a compound or pharmaceutical composition containing the same can be administered and various pK properties are measured in an animal model. In particular, dose-related plasma and brain levels are determined. In some aspects, good brain penetration is about 0.1%, 1%, more than 5%, or more than about 10% of the dose that is able to permeate the blood brain barrier after a given period of time.

Inhibitors of ACAT1 activity have been described. See, e.g., inhibitors listed in Table 1. For example, Ikenoya, et al. ((2007) Atherosclerosis 191:290-297) teach that K-604 has an IC₅₀ value of 0.45 μmol/L for human ACAT1 and 102.85 μmol/L for human ACAT2. As such K-604 is 229-fold more selective for ACAT1 than ACAT2. In addition, diethyl pyrocarbonate has been shown to inhibit ACAT1 with 4-fold greater activity (IC₅₀=44 μM) compared to ACAT2 (IC₅₀=170 μM) (Cho, et al. (2003) Biochem. Biophys. Res. Comm. 309:864-872). Ohshiro, et al. ((2007) J. Antibiotics 60:43-51) teach selective inhibition with beauveriolides I (0.6 μM vs. 20 μM) and III (0.9 μM vs. >20 μM) for ACAT1 over ACAT2. In addition, beauveriolide analogues 258, 280, 274, 285, and 301 show ACAT1 inhibition with pIC₅₀ values in the range of 6 to 7 (Tomoda & Doi (2008) Accounts Chem. Res. 41:32-39). Lada, et al. ((2004) J. Lipid Res. 45:378-386) teach a Warner-Lambert compound (designated therein as Compound 1A), and derivatives thereof (designated Compounds 1B, 1C, and 1D), which inhibit ACAT1 more efficiently than ACAT2 with IC₅₀ values 66- to 187-fold lower for ACAT1 than for ACAT2 (see Table 1). Moreover, Lee, et al. ((2004) Bioorg. Med. Chem. Lett. 14:3109-3112) teach methanol extracts of Saururus chinensis root that contain saucerneol B and manassantin B for inhibiting ACAT activity. Saucerneol B inhibited hACAT-1 and hACAT-2 with IC₅₀ values of 43 and 124 μM, respectively, whereas manassantin B inhibited hACAT-1 and hACAT-2 with IC₅₀ values of 82 μM and only 32% inhibition at 1 mM, respectively.

TABLE 1

| | | IC₅₀ | | | |
|---|---|---|---|---|---|
| Inhibitor | Structure | ACAT1 | | ACAT2 | |
| K-604 | 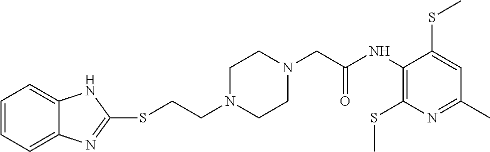 [SMILES: CSc1cc(C)nc(SC)c1NC(=O)CN1CCN(CCSc2nc3ccccc3[nH]2)CC1] 2-[4-[2-(benzimidazol-2- ylthio)ethyl]piperazin-1y1]-N- [2,4-bis(methylthio)-6-methyl-3- pyridyllacetamide | 0.45 | μmol/L | 103 | μmol/L |
| Beauverio- lide I | 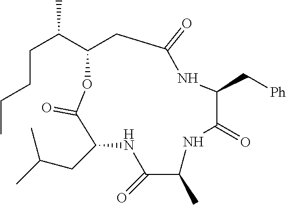 [SMILES: CCCC[C@H](C)[C@@H]1CC(=O)N[C@@H](Cc2ccccc2)C(=O)N[C@@H](C)C(=O)N[C@H](CC(C)C)C(=O)O1] | 0.6 | μM | 20 | μM |
| Beauverio- lide III | 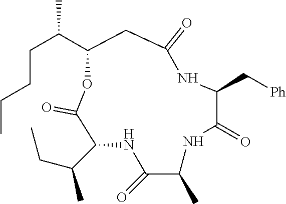 [SMILES: CCCC[C@H](C)[C@@H]1CC(=O)N[C@@H](Cc2ccccc2)C(=O)N[C@@H](C)C(=O)N[C@H]([C@@H](C)CC)C(=O)O1] | 0.9 | μM | >20 | μM |
| Eflucimibe (F12511) | 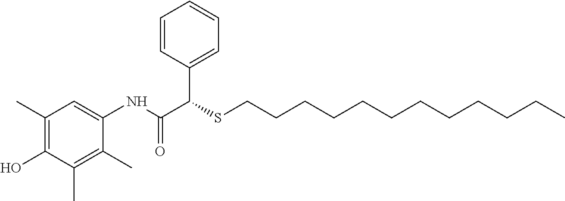 [SMILES: CCCCCCCCCCCCS[C@H](C(=O)Nc1cc(C)c(O)c(C)c1C)c1ccccc1] | 39 | nM | 110 | nM |
| Compound 1A | 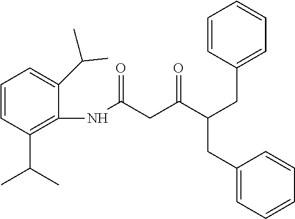 [SMILES: CC(C)c1cccc(C(C)C)c1NC(=O)CC(=O)C(Cc1ccccc1)Cc1ccccc1] | 4.2 | nM | 275 | nM |
| Compound 1B | 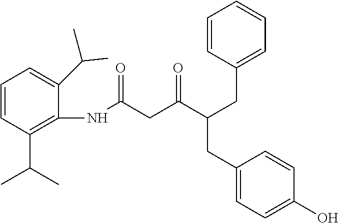 [SMILES: CC(C)c1cccc(C(C)C)c1NC(=O)CC(=O)C(Cc1ccccc1)Cc1ccc(O)cc1] | 10.3 | nM | 1500 | nM |
| Compound 1C | 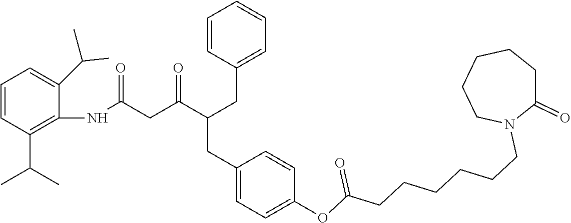 [SMILES: CC(C)c1cccc(C(C)C)c1NC(=O)CC(=O)C(Cc1ccccc1)Cc1ccc(OC(=O)CCCCCCN2CCCCCC2=O)cc1] | 3.6 | nM | 530 | nM |
| Compound 1D | 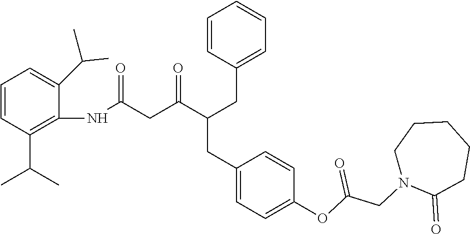 [SMILES: CC(C)c1cccc(C(C)C)c1NC(=O)CC(=O)C(Cc1ccccc1)Cc1ccc(OC(=O)CN2CCCCCC2=O)cc1] | 3.2 | nM | 600 | nM |
| 1^{a} | 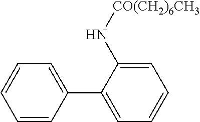 | 61 | μM | 230 | μM |
| 2ª | 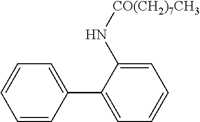 | 65 | μM | 414 | μM |
| 13^{a} | 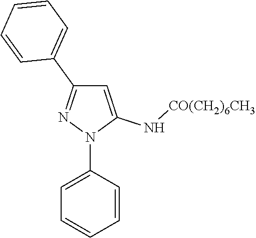 | 24 | μM | 53 | μM |
| 14ª | 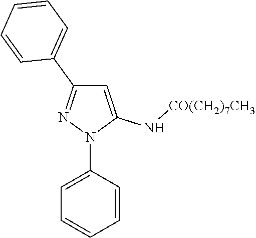 | 23 | μM | 75 | μM |
| 16^{a} | 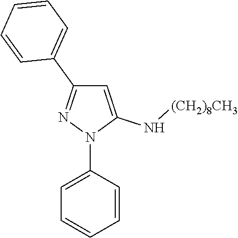 | 39 | μM | 97 | μM |

^{a}Gelain (Jun. 2006) 10th ISCPP, Strasbourg, France.

Desirably, an ACAT1 inhibitor useful in the method of the present invention has an IC₅₀ value in the range of 1 nM to 100 μM. More desirably, the ACAT1 inhibitor has an IC₅₀ value less than 100 μM, 50 μM, 10 μM, or 1 μM. Most desirably, an ACAT1 inhibitor has an IC₅₀ value in the nM range (e.g., 1 nM to 999 nM, or more preferably 1 nM to 50 nM) for human ACAT1.

While the nanoparticle can be composed solely of a pegylated phospholipid, phosphatidylcholine and an ACAT inhibitor, the nanoparticle may further be modified to include a targeting ligand. By “targeting ligand” is meant a molecule that targets a physically associated molecule or complex to a targeted cell or tissue. As used herein, the term “physically associated” refers to either a covalent or non-covalent interaction between two molecules. A “conjugate” refers to the complex of molecules that are covalently bound to one another. For example, the complex of a lipid covalently bound to a targeting ligand can be referred to as a lipid-targeting ligand conjugate.

Alternatively, the targeting ligand can be non-covalently bound to a lipid. “Non-covalent bonds” or “non-covalent interactions” do not involve the sharing of pairs of electrons, but rather involve more dispersed variations of electromagnetic interactions, and can include hydrogen bonding, ionic interactions, Van der Waals interactions, and hydrophobic bonds.

Targeting ligands can include, but are not limited to, small molecules, peptides, lipids, sugars, oligonucleotides, hormones, vitamins, antigens, antibodies or fragments thereof, specific membrane-receptor ligands, ligands capable of reacting with an anti-ligand, fusogenic peptides, nuclear localization peptides, or a combination of such compounds. Non-limiting examples of targeting ligands include transferrin, OX26, melanotransferrin, insulin, ApoE, leptin, thiamine or vitamin B12, rabies virus glycoprotein (RVG), cell penetrating peptides such as TAT peptide, and monoclonal and polyclonal antibodies directed against cell surface molecules. The targeting ligand can be covalently bound to the lipids of the nanoparticle using techniques known in the art (Ishida et al. (1999) FEBS Lett. 460:129-133; Perouzel et al. (2003) Bioconjug. Chem. 14:884-898).

The ACAT1 inhibitor is employed in the methods of the invention for reducing or attenuating neuroinflammation, amyloidopathy or tauopathy and/or preventing or treating conditions resulting from or associated with said neuroinflammation or aberrant amyloid or tau. Neuroinflammation is inflammation of the nervous system, including the central nervous system (CNS). Neuroinflammation can be acute, such as an infection or traumatic event, or chronic, such as a neurodegenerative disease (including demyelinating diseases such as Alzheimer's disease (AD), Parkinson's disease (PD), and multiple sclerosis (MS)). In the CNS, glial cells, including microglia and astrocytes, play an important role in innate immunity. Among other cell types in the brain, these cells can produce cytokines and chemokines that act as neuromodulators. The production of pro-inflammatory compounds can cause neurotoxicity and can compromise the integrity of the blood brain barrier (BBB). Accordingly, use of one or more selective inhibitors of ACAT1 can decrease or eliminate expression of pro-inflammatory response genes (e.g., IL-1β, iNOS, MCP1, Cxc19, Cxc110 and IL-6) and/or increase or enhance expression of anti-inflammatory response genes (e.g., YM1) thereby providing a benefit to a subject with neuroinflammation.

Tau is a highly soluble protein and is associated with microtubules. It can dimerize, oligomerize, then aggregate in vivo. Oligomerized tau is suggested to be the toxic species that causes tauopathy. Tau oligomerization can occur without hyperphosphorylation and when it is associated with the microtubules. Tau is phosphorylated at different sites. Phosphorylation at the 231st threonine, recognized by the specific antibody Thr231, occurs before aggregation. This phosphorylation diminishes the ability of tau to bind to microtubules. Phosphorylation at Ser202 and Thr205 occurs when tau is aggregated, and has been used as a marker for late-stage tau aggregation. Nanoparticle encapsulated inhibitor (F12511 or F26) reduces total unaggregated human tau (htau) as well as aggregated and hyperphosphorylated htau, as determined using antibody AT8, which recognizes phosphorylated Ser202 and Thr205.

Generally, the methods of the invention involve administering to a subject in need of treatment the ACAT1 inhibitor encapsulated in a stealth nanoparticle in an amount that effectively reduces the activity of ACAT1 by at least 60%, 70%, 80%, 90%, 95%, 99% or 100%. Subjects benefiting from treatment with an agent of the invention include subjects exhibiting signs or symptoms of neuroinflammation, amyloidopathy and/or tauopathy or having a condition associated with or resulting from neuroinflammation or aberrant amyloid or tau. Subjects benefiting from treatment with an agent of the invention include those with a neurodegenerative disease (e.g., AD, tauopathy, vascular dementia, PD, Huntington's disease (HD), frontotemporal dementia, amyotrophic lateral sclerosis), neuroinflammatory disorder (e.g., acute disseminated encephalomyelitis, acute optic neuritis, transverse myelitis or neuromyelitis optica), traumatic brain injury or glioma. In the context of this invention, a subject can be any mammal including human, companion animals (e.g., dogs or cats), livestock (e.g., cows, sheep, pigs, or horses), or zoological animals (e.g., monkeys). In particular aspects, the subject is a human.

For therapeutic use, nanoparticle-encapsulated ACAT1 inhibitor can be formulated with a pharmaceutically acceptable carrier at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, P A, 2000. A pharmaceutically acceptable carrier, composition, or vehicle, such as a liquid or solid filler, diluent, excipient, or solvent encapsulating material, is involved in carrying or transporting the subject agent from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Administration can be parenteral (for example, by intravenous, intraperitoneal, subcutaneous, or intramuscular injection), topically, orally, intranasally, intravaginally, or rectally according to standard medical practices. Alternatively, the composition can be administered directly to the brain of the subject by a shunt or a catheter. In particular aspects, the pharmaceutical composition is formulated for intravenous or intraperitoneal delivery.

In certain aspects of the present invention, the encapsulated ACAT1 inhibitor is selectively delivered to the brain. For the purposes of the present invention, “selective delivery to the brain” or “selectively delivered to the brain” is intended to mean that the agent is administered directly to the brain of the subject (e.g., by a shunt or catheter; see, e.g., US 2008/0051691), to the perispinal space of the subject without direct intrathecal injection (see, e.g., U.S. Pat. No. 7,214,658), or in a form which facilitates delivery across the blood brain barrier thereby reducing potential side effects associated with ACAT1 inhibition in other organs or tissues. In this regard, formulation of the agent into a nanoparticle in the presence of a stabilizer allows passage of the blood brain barrier without affecting other organs (producing toxicity). See, e.g., U.S. Pat. No. 7,402,573, incorporated herein by reference in its entirety. Furthermore, an exemplary system for selectively delivering microRNAs to the brain is the Adeno-Associated Virus (AAV) vector system. See, e.g., Cearley & Wolfe (2007) J. Neurosc. 27(37):9928-9940.

It has been shown that exosomes (i.e., natural transport nanovesicles in the range of 40-100 nm), which express Lamp2b fused to the neuron-specific rabies viral glycoprotein (RVG) peptide, can deliver siRNA specifically to neurons, microglia, and oligodendrocytes in the brain, thereby resulting in specific gene knockdown (Alvarez-Erviti, et al. (2011) Nature Biotechnol. 29:341-345). Accordingly, in an alternative aspect, the ACAT1 inhibitor is delivered to the brain via an exosome, in particular an exosome modified with a moiety that targets cells of the brain. Exosomes of use in this invention can be prepared by methods such as those described by, e.g., Sun, et al. (2010) Mol. Ther. 18:1606-1614. Likewise, therapeutic agents can be encapsulated within exosomes by methods such as incubating the therapeutic agent with an exosome preparation in saline at room temperature for several minutes, and separating the exosomes from unencapsulated drug and debris, e.g., by sucrose gradient separation. As described in the art, moieties that target cells of the brain include peptides that target cells of the brain (e.g., neurons, microglia and/or oligodendrocytes) as well as other targeting agents such as lipopolysaccharide, which has a high affinity for surface markers on microglia (Chow, et al. (1999) J. Biol. Chem. 274:10689-10692). Targeting peptides include, e.g., the RVG peptide, which may be fused to membrane bound proteins, e.g., Lamp2b (Lysosome-associated membrane protein 2b) to facilitate integration into the exosome. Moreover, when the agent is a nucleic acid (e.g., siRNA or miRNA), the targeting peptide can be fused with a polyarginine peptide (e.g., nine D-arginines) so that the nucleic acid is electrostatically bound to the targeting moiety. In addition to using exosomes for delivery of the compositions, one of skill would understand that untargeted or brain-targeted liposome has been used successfully to facilitate delivery of the siRNA or small molecule inhibitors to brain tissue (Pardridge (2007) Adv. Drug Deliv. Rev. 59:141-152; Pulford, et al. (2010) PLoS ONE 5:e11085). As a result, aspects of the methods of the present invention include using of liposomes that are either targeted or untargeted.

In another alternative aspect, the ACAT1 inhibitor is delivered intranasally via an exosome. Curcumin or Stat3 inhibitor, JSI-124 (cucurbitacin I), delivered via exosomes to the brain via the nasal route has been shown to accumulate in microglia and inhibit lipopolysaccharide (LPS)-induced microglial cell activation, delay experimental autoimmune encephalomyelitis (EAE) disease, and inhibit tumor growth in vivo (Zhuang, et al. (2011) Mol. Ther. 19:1769-1779). It is posited that transport occurs along the olfactory pathway and likely involves extracellular bulk flow along perineuronal and/or perivascular channels, which allows for delivering drugs directly to the brain parenchyma. Delivery along the extraneuronal pathway is likely not receptor-mediated and requires only minutes for a drug to reach the brain; whereas, delivery via an intraneuronal pathway along the primary olfactory sensory neurons involves axonal transport and requires several days for the drug to reach different areas of the brain. Therefore, in certain aspects, the ACAT1 inhibitor of the invention is delivered to the brain, in particular microglia, by encapsulating within exosomes and intranasal administration.

For encapsulation in a nanoparticle, an ACAT inhibitor is prepared at a concentration of 10 mM. Once injected into the blood, its concentration is expected to be diluted to approximately 1 mM. Based on HPLC quantitation analyses of F12511 in the blood, F12511 undergoes degradation more rapidly than that of the nanoparticle. However, its concentration in the blood is high enough that a substantial portion of F12511 (approximately 0.1% to 0.2%) enters the brain to provide significant inhibition of ACAT activity in the brain. Indeed, as demonstrated herein, a DSPE-PEG₂₀₀₀- and phosphatidylcholine-based nanoparticle with a high concentration of ACAT1 inhibitor encapsulated in the nanoparticles, provided a high level of ACAT1 inhibitor in the blood, entered the CNS, and significantly inhibited ACAT1 in the brain for a prolonged period of time (longer than 8 hours). Thus, in certain aspects, the ACAT1 inhibitor is encapsulated in a nanoparticle that maintains a concentration of the inhibitor in the blood of the subject at above 20 μM for at least 4 to 8 hours, thereby providing a therapeutic benefit to a patient.

In a further alternative aspect, the ACAT1 inhibitor is encapsulated in human serum albumin (HSA) nanoparticles to enhance solubility and bioavailability of the inhibitor. Such nanoparticles can be fabricated via unfolding of HSA in appropriate solution to expose more hydrophobic domains and consequent self-assembling into nanoparticles with the ACAT inhibitor. See Zhou, et al. (2016) Anticancer Res. 36(4):1649-56; Ding, et al. (2014) AAPS PharmSciTech. 15(1):213-22.

To enhance treatment of one or more of the disorders or conditions described herein, the ACAT1 inhibitor may be administered in combination with a second therapeutic agent that complements the action of the ACAT1 inhibitor. Ideally, the second therapeutic agent may target a cause of the underlying disease or condition, e.g., misfolded/aggregated protein/peptide such as amyloid B in AD, tau in AD, α-synuclein in PD, and frontotemporal dementia, and huntington in HD. Examples of second therapeutic agents of use in combination with the ACAT1 inhibitor include, but are not limited to, β-secretase inhibitors/modulators (e.g., AZD3293, CTS-21166, E2609, HPP854, LY2886721, MK-8931, PF-05297909, RG7129 or TK-070), γ-secretase modulators (e.g., LY-411575, LY-450139, begacestat, ELN-475516, BMS-708163, MRK-003, CHF5074 or R04929097), proteasome inhibitors (e.g., Bortezomib), small molecule activators of the unfolded protein response (e.g., Celastrol), Hsp90 inhibitors (e.g., Geldanamycin), small molecule Hsc70 inhibitors (e.g., YM-01), deubiquitination enzyme inhibitors (e.g., siRNA targeting Usp14), epigallocatechin-3-gallate, variants of Hsp104 disaggregase, glutathione ethyl ester, and antibodies (e.g., anti-oligomeric amyloid B antibody, anti-tau antibody, anti-α-synuclein antibody or anti-huntington antibody). When included, the second therapeutic agent can be co-administered as a conventional pharmaceutical composition along with the nanoparticle encapsulated ACAT inhibitor, administered in the same nanoparticle as the ACAT inhibitor, or co-administered in a separate nanoparticle along with the nanoparticle encapsulated ACAT inhibitor. As with the ACAT inhibitor, administration of the second therapeutic agent via a nanoparticle will allow for the second therapeutic agent to enter the brain interior at concentrations sufficient to provide therapeutic benefit to the patient.

The selected dosage level of an ACAT1 inhibitor and optional second therapeutic agent will depend upon a variety of factors including the activity of the particular agent of the present invention employed, the route of administration, the time of administration, the rate of excretion or metabolism of the particular agent being employed, the duration of the treatment, other drugs, compounds and/or materials used in combination with the particular agent employed, the age, sex, weight, condition, general health and prior medical history of the patient being treated, and other factors well-known in the medical arts.

A physician or veterinarian having ordinary skill in the art can readily determine and prescribe the effective amount of the pharmaceutical composition required based upon the administration of similar compounds or experimental determination. For example, the physician or veterinarian could start doses of an agent at levels lower than that required in order to achieve the desired therapeutic effect and gradually increase the dosage until the desired effect is achieved. This is considered to be within the skill of the artisan and one can review the existing literature on a specific agent or similar agents to determine optimal dosing.

The method for reducing or attenuating amyloidopathy or tauopathy comprises administering to a subject in need thereof a nanoparticle having a core and an outer lipid envelope, wherein the outer lipid envelope is composed of DSPE-PEG and PC. The DSPE-PEG/PC-based nanoparticle may be modified to include one or more additional lipids, e.g., sphingomyelin, cholesterol, ceramide, cardiolipin, as well as other minor lipid species, to increase potency in reducing or attenuating amyloidopathy and/or tauopathy.

Ideally, the DSPE-PEG to PC ratio is in the range of 30:3 to 30:12, or more preferably 30:6. Moreover, in certain aspects, the concentration of PC is greater than or equal to 6 mM. Advantageously, nanoparticle composed of 30 mM DSPE-PEG and 6 mM PC can provide 3 mM DSPE-PEG and 0.6 mM PC in the blood of a patient immediately upon delivery of the nanoparticle and provide therapeutic benefit by reducing or attenuating amyloid burden and aggregated and hyperphosphorylated htau but not total unaggregated human tau.

The invention is described in greater detail by the following non-limiting examples.

### Example 1: Material and Methods

Lipids, ACAT Inhibitors, and Solvents: DSPE-PEG₂₀₀₀ was from Laysan Bio, Inc (mPEG-DSPE, MW 2,000). L-α-Phosphatidylcholine was from Sigma-Aldrich. F12511 and K-604 were custom synthesized by WuXi AppTec in China. Based on HPLC/MS and NMR profiles, purity of F12511 was 98% and in stereospecificity; purity of K-604 was 98% in chemical purity. Organic solvents were from Fisher Scientific.

Mice. Mice were fed ad libitum with standard chow diet, maintained in a pathogen-free environment in single-ventilated cages and kept on a 12-hour light/dark schedule.

Generation of Acat1^{-/-} Alz (A1⁻/Alz) and Acat2^{-/-} Alz (A2⁻/Alz) Mice. Acat1^{-/-} and Acat2^{-/-} mice (Meiner, et al. (1996) Proc. Natl. Acad. Sci. USA 93:14041-14; Buhman, et al. (2000) Nat. Med. 6:1341-1347) in C57BL/6 background are known in the art. The 3×Tg-Alz mice (Alzheimer's disease mice) in hybrid 129/C57BL/6 background contain two mutant human transgenes, hAPP harboring Swedish mutation (hAPPswe), and mutant htau (htau_{P301L}) under a neuron-specific promoter, and contain the knock-in mutant presenilin 1 (PS1_{M146V}) (Oddo, et al. (2003) Neuron 39:409-421).

Cell Culture. Cell lines were kept in a 37° C. incubator with 5% CO₂. Human neuron-like SH-SY5Y cells were maintained in 1:1 EMEM (Corning, Manassas, VA):Ham's F-12 (Sigma, St. Louis, MO) medium supplemented with MEM non-essential amino acids (Gibco, Grand Island, NY) and 10% calf serum (R&D Systems, Flowery Branch, GA). Human microglia-like HMC3 cells (ATCC) were maintained in MEM (Corning, Manassas, VA) medium with 10% calf serum. Mouse neuron-like N2a cells were maintained in 1:1 DMEM (Corning, Manassas, VA):MEM (Corning, Manassas, VA) medium with 10% calf serum. Mouse microglia-like N9 cells were maintained in RPMI (Corning, Manassas, VA) medium with 10% calf serum.

Primary cortical neurons were dissected from E14.5-16.5 embryos from C57/BL6 mice and plated at 50,000 cells per well in precoated poly-D-lysine (Sigma-Aldrich, St. Louis, MO) and laminin-coated (Sigma-Aldrich, St. Louis, MO) 96-well tissue culture dishes. Cells were incubated in enriched neurobasal (NB) medium (Thermo Fisher Scientific, Waltham, MA) (500 mL NB media, 200 mM L-glutamine, B-27 with vitamin A, 1 mL penicillin-streptomycin (1%), and 340 mg glucose). Neurons were allowed to grow processes for 4-6 days prior to treatment.

Isolation of Microglia from Adult Mouse Brain. Microglia cells were isolated from adult mouse brains using CD11b MicroBeads (Miltenyi Biotec, San Diego, CA) as described previously (Nikodemova & Watters (2012) J. Neuroinflamm. 9:147). The CX3CR1/GFP^{+/+} mice were used to examine the purity of microglia; GFP expression within the central nervous system of this mouse line is almost exclusively in microglia (Jung et al. (2000) Mol. Cell. Biol. 10:4106-14).

F12511 LC-MS/MS Protocol. F12511 and internal standard CP113,818 were quantified in mouse tissues via LC-MS/MS. Both compounds were dissolved in DMSO at 10 mg/mL and stored at −40° C. Subsequent working dilutions were made in acetonitrile (ACN) fresh daily. Calibrators and quality controls were made in the appropriate matrix: C57BL6 plasma (anticoagulant: K3-EDTA, Innovative Research), C57BL6 whole blood (anticoagulant: K3-EDTA, Innovative Research), C57BL6 brain homogenate (study provided), or C57BL6 liver homogenate (study provided). Tissues were homogenized at 0.1 g/mL in diH₂O using stainless steel beads and a Next Advance Bullet Blender. All samples (50 μL) were protein precipitated with 150 μL 10 ng/mL CP113,818 in ACN via vortex 1 minute and centrifugation 5 minutes at 15,000 rpm. 150 μL of supernatant was transferred to amber autosampler vials and 10 μL was injected on the LC-MS/MS system.

HPLC separation was achieved with isocratic conditions of 5% diH₂O, 95% methanol, and 0.1% formic acid over 2.5 minutes at a flow rate of 1.5 mL/min on a Phenomenex Luna C18 100×4.6 mm, 3 micron column fitted with a 10×4 mm C18 guard at 40° C. A TSQ Vantage mass spectrometer was operated in positive ion mode with a collision pressure of 1.3 mTorr to measure F12511 (470.242->268.120 m/z) and CP113,818 (471.177->201.040 m/z) with collision energies of 16 and 21, and SLens values of 139 and 187, respectively. The heated ESI source was operated with a spray voltage of 4500 V, vaporizer temperature 500° C., capillary temperature 250° C., sheath and auxiliary gases at 30 and 15 arbitrary units, respectively. The quantitative range was 0.3-1000 ng/mL for liver, brain, and whole blood, and 0.5-1000 ng/mL for plasma with the following inter- and intraday confidence values (CV) and accuracies (Ac) of across three quality control levels: plasma intraday CV 3-14%, Ac 86-109%; plasma interday CV 13-15%, Ac 95-100%; liver intraday CV 2-10%, Ac 93-114%; liver interday CV 5-8%, Ac 94-111%; whole blood intraday CV 3-10%, Ac 85-115%; whole blood interday CV 12-14%, Ac 96-115%; brain intraday CV 2-11%, Ac 94-115%; brain interday CV 9-14%, Ac 96-108%.

Nanoparticle Formation. Nanoparticles were prepared according to a general method (Gülçür, et al. (2013) Drug Deliv. Transl. Res. 3(6):562-574; Jhaveri & Torchilin (2014) Front. Pharmacol. 5:77) with modifications. Using a clean glass tube (9 mL capacity), 60 mg of DSPE-PEG₂₀₀₀ was dissolved in 500 μL of ethanol (EtOH) for a working concentration of 60 mM. Phosphatidylcholine (PC) (dissolved in chloroform) was added at working concentrations ranging from 0 to 12 mM to the DSPE-PEG₂₀₀₀ solution while vortexing. F12511 was dissolved in 500 μL of ethanol at working concentrations of 12-24 mM then added to the DSPE-PEG₂₀₀₀/PC mixture while vortexing. The final solution contained concentrations of 30 mM DSPE-PEG₂₀₀₀, 0-6 mM PC, and 6-12 mM F12511. The final solution was then lyophilized under refrigeration overnight to remove organic solvent. The sample was re-suspended in 1 mL of phosphate-buffered saline (1×) (PBS) and vortexed until the sample was in suspension. This step took approximately 1 hour as the sample needed to rest between repeated vortexing to prevent excessive foaming. Once in fine suspension, the sample was purged with nitrogen, capped, wrapped with parafilm, and bath sonicated (Branson 2510 model) at 4° C. for 2-4 rounds at 20 minutes per round. DSPE-PEG₂₀₀₀/PC nanoparticles without F12511 were clear while F12511/DSPE-PEG₂₀₀₀/PC nanoparticles (Nanoparticle F) were slightly turbid and contained some visible precipitate. After sonication, nanoparticles were transferred to sterile Eppendorf tubes and centrifuged at 12,000 rpm for 5 minutes to remove unincorporated materials. The supernatant as well as the pellet were collected for chemical analysis. In some experiments, the nanoparticles were loaded onto a 5 mL SEPHAROSE® (crosslinked agarose) CL-4B column. SEPHAROSE® CL-4B contains beads with particle size at 45-165 μm. The column (approximately 28 mm circumference) was first equilibrated with PBS at room temperature, then loaded with the 1 mL of sample, eluted with PBS and fractions of 500 μL to 1 mL were collected. This method was used to assure that F12511 or K-604 encapsulated in nanoparticles did not appear in the exclusion volume of the SEPHAROSE® CL-4B column.

Nanoparticle Analysis. To confirm the concentration of the ACAT inhibitor (i.e., F12511 or K-604) and DSPE-PEG₂₀₀₀/PC in the nanoparticles, 10 μL of each sample component were loaded onto a thin layer chromatography (TLC) plate (Analtech Silica gel HL). The solvent systems used were hexanes:ethyl ether:acetic acid (60:40:1) (Bryleva, et al. (2010) Proc. Natl. Acad. Sci. USA 107(7):3081-3086) to detect F12511 and chloroform:methyl acetate:isopropanol:methanol:water (28:25:25:12:10) to detect K-604. The retention factor (Rf) was approximately 0.35 for F12511 and approximately 0.5 for K-604. The content of F12511 or K-604 was determined by extrapolation from a standard curve of a concentration gradient of the respective compound produced in the same TLC plate. The plate was stained with iodine and quantified using ImageJ. The TLC/iodine stain method detected F12511 or K-604, and DSPE-PEG₂₀₀₀/PC separately, thus allowing for encapsulation efficiency determination.

Dynamic Light Scattering (DLS) Measurements. Nanoparticle sizes were measured with Malvern Panalytical Zetasizer Nano-ZS, using 10 mm path length disposable cuvettes. Samples were kept in 4° C. in tubes wrapped with parafilm and left in the dark whenever possible. Prior to analysis, samples were centrifuged for 5 minutes at 12,000 rpm. Most samples required 2-fold dilution to get an accurate reading. Samples were measured in triplicate.

Intact Cell Cholesteryl Esterification by ³H-Oleate Pulse. Using an established procedure (Chang, et al. (1986) Biochemistry 25(7):1693-1699), monolayers of cells were treated for 2 hours with various concentrations of either control (ethanol or PBS), DSPE-PEG₂₀₀₀/PC nanoparticles, F12511 (dissolved in ethanol), or F12511/DSPE-PEG₂₀₀₀/PC (Nanoparticle F) nanoparticles. Following treatment, cells were pulsed at 37° C. for 1-3 hours with ³H-oleate/fatty acid free BSA, then washed with cold PBS, lysed by adding cold 0.2 M NaOH at appropriate volume, and placed on orbital shaker for 40 minutes. The solubilized cell slurries were collected into glass tubes, neutralized with 3M HCl, and lipids were extracted with CHCl₃:MeOH (2:1) and water. Samples were vortexed and centrifuged at 500 rpm for 10 minutes. The top-phase was removed, and the bottom phase was blown dry with N₂ using N-evap apparatus (Organomation Associates, Inc.). Dried samples were vortexed with ethyl acetate and spotted on TLC plate. The solvent system used was petroleum ether:ethyl ether:acetic acid (90:10:1). The cholesteryl ester band was scraped from the TLC plate and measured with a scintillation counter for radioactivity.

In vitro Mixed Liposomal ACAT Activity Assay. Using an established procedure (Chang, et al. (1998) J. Biol. Chem. 273(52):35132-35141; Neumann, et al. (2019) Arch. Biochem. Biophys. 671:103-110). Monolayers of cells were treated for 4 hours with either control (dimethyl sulfoxide (DMSO)), or with 0.5 μM F12511 in DMSO. Cells were then washed with drug-free medium kept at 37° C. for various amounts of time. At the indicated times, cells were washed with cold PBS, then lysed in a 2.5% 3-((3-Cholamidopropyl) dimethylammonio)-1-propanesulfonate (CHAPS), 1M KCl in 50 mM Tris at pH 7.8 buffer. Lysed cells were collected and aliquoted into pre-chilled tubes containing the liposomal mixture of taurocholate, phosphatidylcholine, and cholesterol. Tubes were vortexed and kept on ice for 5 minutes. Samples were then incubated in a 37° C. shaking water bath with ³H-oleoyl CoA added for 10 minutes. The assay was stopped by adding CHCl₃:MeOH (2:1). Lipids were extracted and analyzed by the same TLC method described herein.

Lactate Dehydrogenase (LDH) Toxicity Assay. Primary cortical neurons were harvested and maintained as described above. Neurons were treated with either F12511 alone (dissolved in EtOH), F12511/DSPE-PEG₂₀₀₀/PC nanoparticles (Nanoparticle F), or DSPE-PEG₂₀₀₀/PC nanoparticles at 2-100 μM concentrations for 24-, 48-, or 72-hours. After treatment, the conditioned media from the cells were collected, spun at 12,000 rpm for 30 minutes to remove cellular debris, supernatant transferred to new tube, and frozen at −80° C. until time of assay. Using Cytotoxicity Detection Kit (LDH) (Roche), media and reaction mixture were mixed, incubated for 30 minutes at room temperature protected from light, and the absorbance was measured at 490 nm.

### Example 2: F26 is a Brain Permeable ACAT Inhibitor

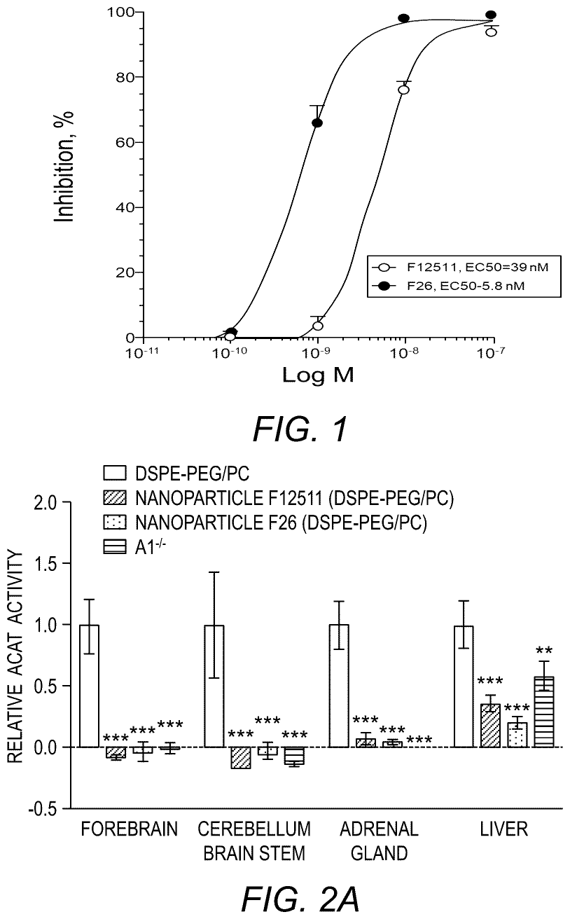

ACAT1 is a drug target for the treatment of AD. To achieve optimal drug treatment, it is important to use a potent ACAT inhibitor that also is capable of permeating the blood-brain barrier and delivering sufficient concentrations of the ACAT inhibitor to brain tissue. It was determined that F26 inhibits ACAT1 and was almost 7 times more potent than F12511. The EC₅₀ values of F12511 and F26 (98% pure) for human ACAT1 were compared using the mixed liposome assay and the results indicated that F26 was almost 7 times more potent than F12511 as an ACAT1 inhibitor (EC50=5.8 nM for F26 versus 39 nM for F12511) (see FIG. 1).

To determine in vivo activity, F12511 and F26 were delivered IV to mice via stealth nanoparticles (nanoparticle F12511 and nanoparticle F26). Adult C57BL/6 mice were given single IV of nanoparticle F12511 (at high dose ˜46 mg/kg) or nanoparticle F26 (at low and high doses). Six hours after IV injection, various tissues were isolated and subjected to ACAT inhibition measurements. The results (FIG. 2A) showed that, in two different regions of the mouse brain, a low dose of F26 (3 mM) provided very similar percent ACAT inhibition (more than 80%) as F12511 when administered at higher doses (9 mM), indicating that F26 is also brain-permeable and is more potent at inhibiting ACAT activity in the brain than F12511. Turning to the high dose of F26 (9.0 mM), it was observed that both nanoparticle F12511 and nanoparticle F26 provided more than 95% enzyme inhibition (FIG. 2B).

### Example 3: A Single Systemic Dose of Nanoparticle F26 Decreases ACAT Enzyme Activity for 48 Hours In Vivo

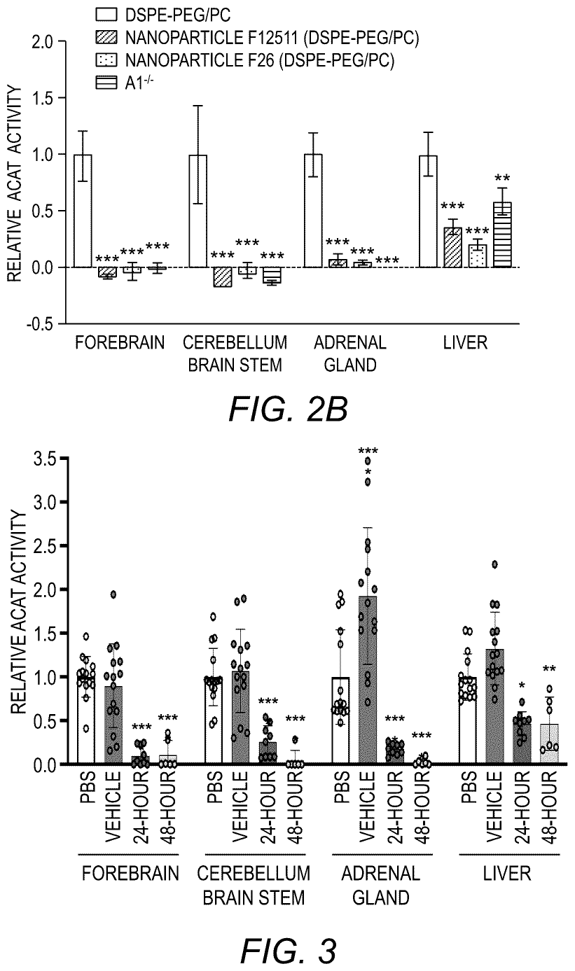

To characterize the duration of nanoparticle F26, a single retro-orbital injection of nanoparticle F26 at 43.7 mg/kg was performed in the wild-type (C57BL/6) mice and the tissues, forebrain, cerebellum/brain stem, adrenal gland, and liver were collected to measure ACAT enzyme activity after 24-48 hours post-injection (n=2-6 mice). No change of ACAT enzyme activity was observed between PBS-injected and nanoparticle vehicle (DSPE-PEG/PC)-injected groups in tissues of forebrain, cerebellum, and liver. In contrast, ACAT enzyme activities were significantly decreased in forebrain by 90%, cerebellum/brain stem by 74-95%, adrenal gland by 72-97%, and liver by 53%, both after 24- and 48-hours post-injection (FIG. 3). As such, these results demonstrate that nanoparticle F26 can sustain ACAT inhibition for 48 hours.

### Example 4: F12511 Encapsulated in DSPE-PEG₂₀₀₀/PC Nanoparticles Significantly Inhibits ACAT Activity in Mouse and Human Neuronal and Microglial Cell Lines

To test the efficacy of the nanoparticles at inhibiting ACAT activity, mouse and human neuronal and microglial cell lines were used. Intact cells were treated with either control (EtOH), DSPE-PEG₂₀₀₀/PC nanoparticles, F12511 alone (dissolved in EtOH), or F12511 DSPE-PEG₂₀₀₀/PC nanoparticles (Nanoparticle F). The results showed that across all cell lines, both F12511 alone and nanoparticle F12511 tested at 0.04 μM and 0.4 μM significantly inhibited ACAT in a dose dependent manner. These results indicated that nanoparticle F12511 was almost as efficient as F12511 alone in inhibiting ACAT in intact cells, even with a short treatment time of 2 hours. Notably, F12511 alone or nanoparticle F12511 was more effective in inhibiting ACAT in human cell lines (SH-SY5Y and HMC3 cells) than in mouse cell lines (N2A and N9 cells).

F12511 inhibits ACAT1 at high affinity with an IC₅₀ value and inhibitory constant (Ki) of 39 nM (Chang, et al. (2000) J. Biol. Chem. 275(36):28083-92). In general, enzyme inhibitors with a low Ki interact with the target enzyme with low dissociation rate. It was posited that once F12511 binds to ACAT1, it may dissociate from the enzyme slowly. This was analyzed at the cellular level by incubating F12511 alone or nanoparticle F12511 in neuronal and microglial cell types. After 2 hours of treatment, media containing F12511 was removed, cells were washed twice with drug-free media, and placed in drug-free media for either 0-, 2-, 4-, or 8-hours. Afterward, the ACAT activity in these cells was measured by performing ³H-oleate pulse. The results showed that in F12511 treated cells, ACAT activity remained significantly inhibited in all cell types examined, even after 8 hours of drug removal from the media. For nanoparticle F12511 treated cells, ACAT activity also was significantly inhibited for considerable time (4-6 hours). At 8 hours, significant inhibition occurred in SH-SY5Y, N2A and N9 cells but not in HMC3 cells. Together, these results indicated that in intact cells, once bound to ACAT1, the ACTA1 inhibitor F12511 slowly dissociates from the enzyme. The validity of these results was analyzed using a different method to monitor ACAT enzyme activity. N9 cells were treated with DMSO as control or with F12511 alone for 4 hours, washed with drug-free media, and incubated in drug-free media for various amounts of time, up to 4 hours. Here, the ACAT activity was monitored using the ACAT enzyme assay in vitro. The in vitro assay monitors the interaction between ACAT and F12511 in the milieu where detergent taurocholate, phospholipid, and cholesterol are present in abundance (Chang, et al. (1998) J. Biol. Chem. 273 (52):35132-35141). The results of this analysis showed that, even under this condition, the ACAT enzyme activity stayed inhibited by F12511 for up to 4 hours after F12511 removal from the media. Together, the results of these analyses indicated that once F12511 binds to ACAT, it does not readily dissociate from the enzyme for several hours and the binding between ACAT1 and F12511 is not readily affected by the presence of cholesterol, phospholipids, or detergent.

### Example 5: ACAT1 Inhibition Suppresses Pro-Inflammatory Gene Expression

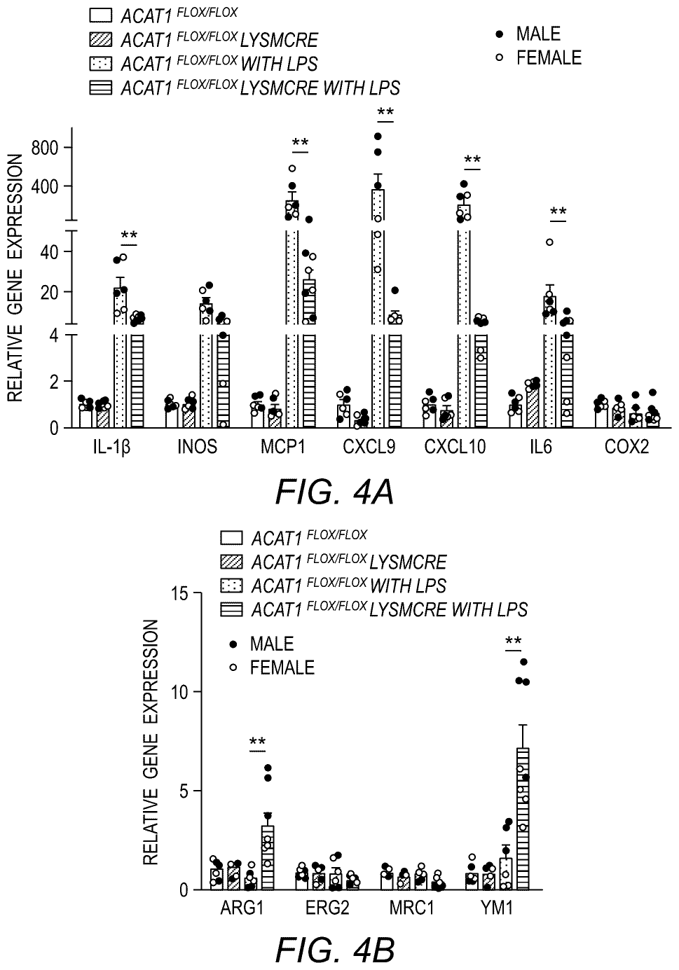

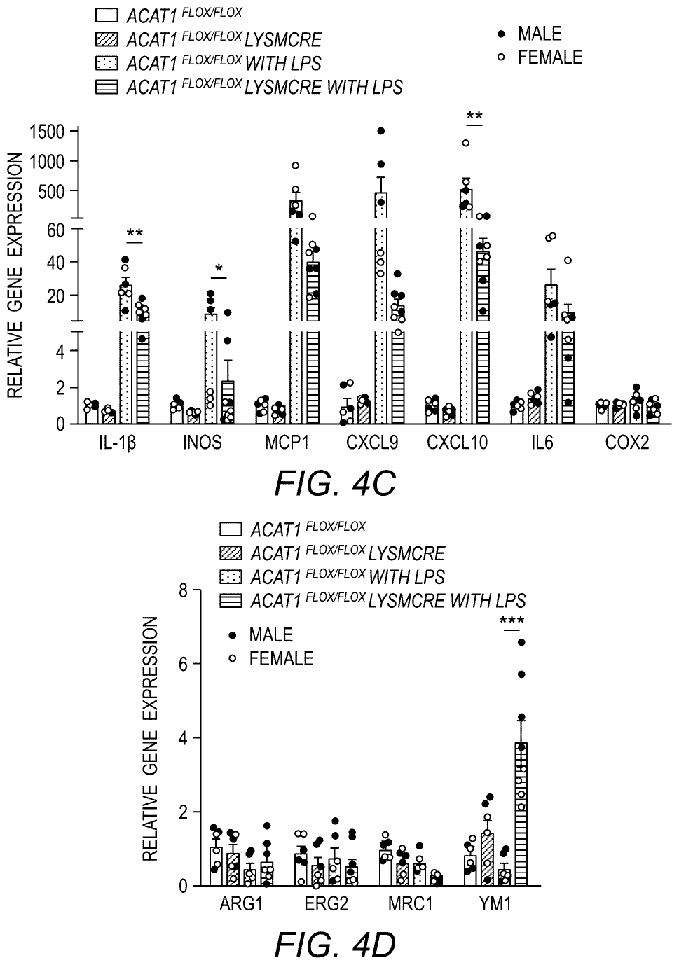

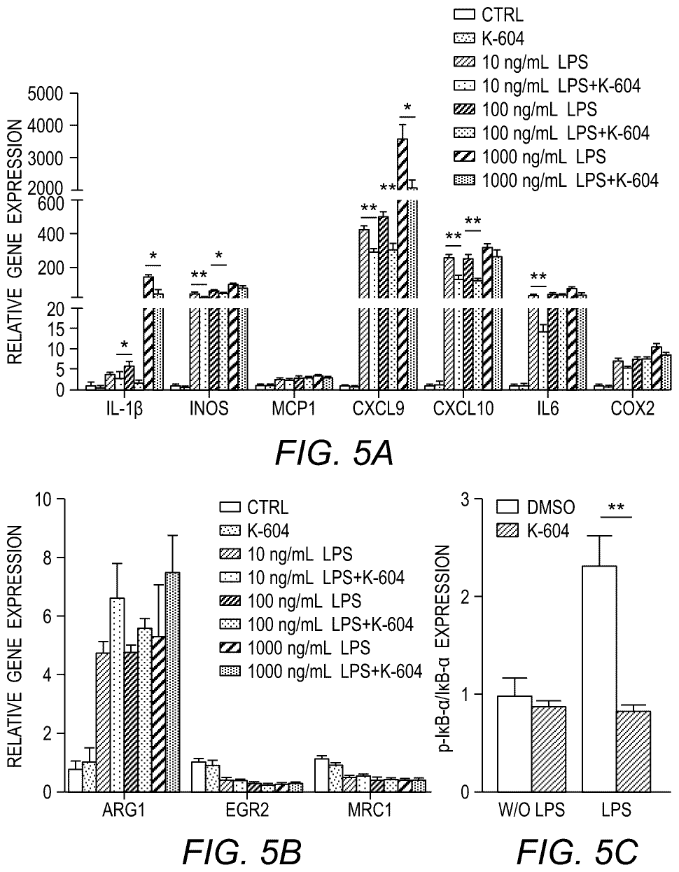

It has now been shown that, in microglia, genetic inactivation of Acat1 suppresses certain pro-inflammatory response genes (FIG. 4A and FIG. 4C, e.g., IL-1β, iNOS, MCP1, Cxc19, Cxc110 and IL-6) and induces certain anti-inflammatory response genes (FIG. 4B and FIG. 4D, e.g., YM1) in the hippocampus (FIG. 4A and FIG. 4B) and cortex (FIG. 4C and FIG. 4D). Likewise, inhibiting ACAT1 protein activity in microglial cells attenuates inflammatory responses to LPS (FIG. 5A-5C). These results indicate that the actions of ACAT1 blockage in microglia involves interleukin 4 (IL-4), which is one of the major anti-inflammatory cytokines. To demonstrate this in microglia, ACAT1 inhibition was shown to increase the protein content of IL4R-α, which is the receptor for IL-4 (FIG. 6).

In microglia, LPS uses Toll-like receptor 4 (TLR4) as one of the main receptors to mediate its inflammatory actions. The lipid efflux protein, ABCA1, is known to suppress pro-inflammatory responses, in part by attenuating the activity of TLR4 at the plasma membrane. Accordingly, it was posited that lipopolysaccharides (LPS) or other TLR4 agonists stimulate the CD36/TLR4-associated complexes at the plasma membrane (PM) and activate the cytosolic IkB kinase, which rapidly phosphorylates IkB (FIG. 5C), leading to its translocation from the cytosol into the nucleus to activate pro-inflammatory gene transcription. IL-4 or IL-13 binds to IL4Rα at the PM, which leads to the rapid phosphorylation of the transcription factor, STAT6, as well as increased expression of other transcription factors, including PPARγ and KLF4. These events increase the expression of anti-inflammatory genes. The ACAT1 blockade leads to accumulation of free cholesterol [and/or regulatory sterols such as 25-hydeoxycholesterol (25HC)], which further leads to an upregulation of IL4Rα to increase the anti-inflammatory cascade, and/or upregulation of ABCA1, and/or downregulation of TLR4, to suppress the pro-inflammatory cascade.

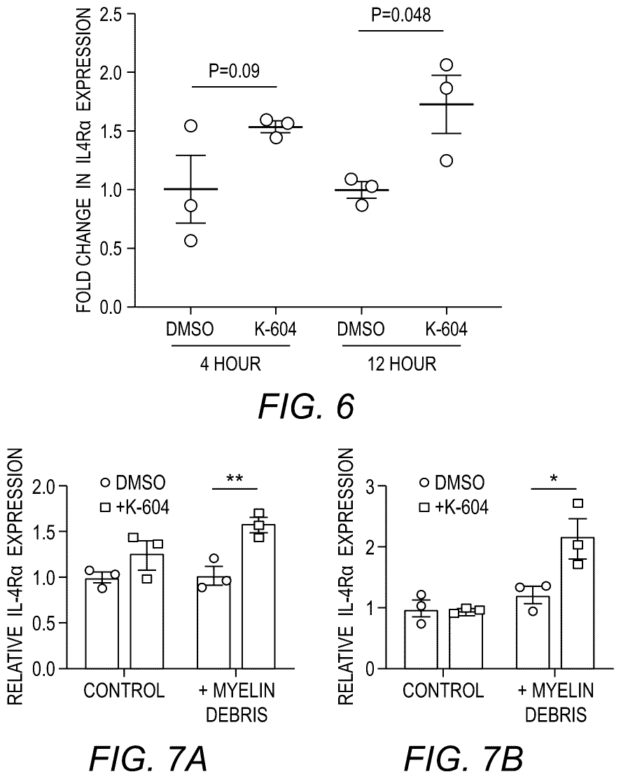

In microglia, various cholesterol-rich agents including myelin debris and damaged cell membranes enter the cells. It was posited that ACAT1 enzyme activity may be upregulated by cholesterol released from these cholesterol-rich agents. Accordingly, N9 cells were treated with myelin debris at two different doses (25 μg cholesterol/mL and 125 μg cholesterol/mL) for 21 hours and it was found that, indeed, myelin debris caused a significant increase in cholesterol ester biosynthesis, in a dose-dependent manner. To demonstrate the specificity of myelin debris in activating cholesteryl ester biosynthesis, N9 cells, when treated with myelin debris, did not exhibit any significant changes in triacylglycerol (TAG) biosynthesis or in phospholipid biosynthesis. In addition, the effects of ACAT1 blockade were tested in N9 and the human microglia-like cell line HMC3, which both express normal TREM-2 receptors. The results showed that, in both N9 cells (FIG. 7A) and HMC3 cells (FIG. 7B), the enhancing effect of ACAT1 inhibition by K-604 on IL4R-α was indeed more robust when cells were incubated with myelin debris in the media.

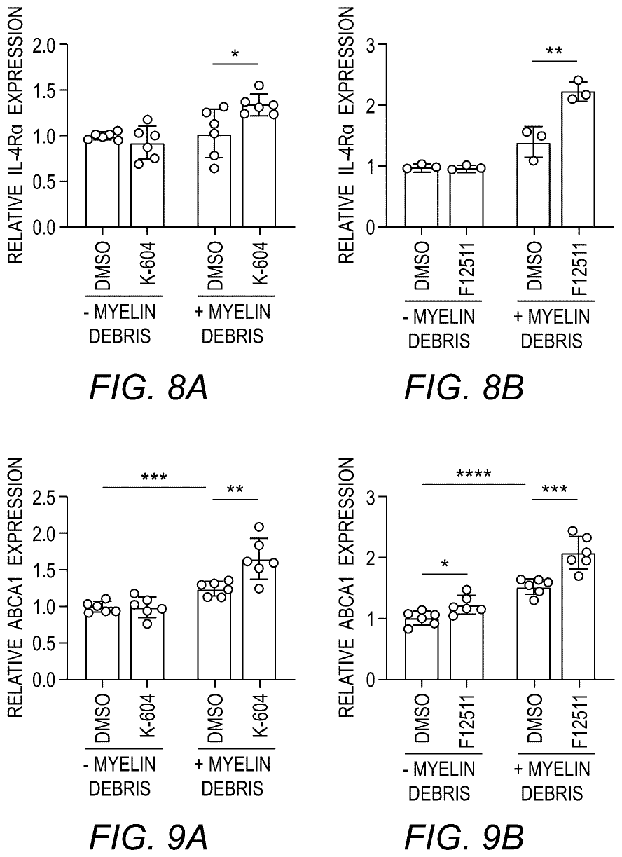

Subsequently, myelin debris from human brains was prepared and it was observed that the cholesterol/protein ratio, as well as the protein composition between mouse myelin debris and human myelin debris, were very similar. In N9 cells, human myelin debris added to the medium at 25 μg/mL cholesterol also significantly increased cholesteryl ester biosynthesis; this synthesis was effectively suppressed by ACAT1 inhibitor K-604. In HMC3 cells, the enhancing effects of K-604 on IL4R-α levels (FIG. 8A) was replicated by F12511 (FIG. 8B). It was also found that the protein content of the key lipid efflux protein, ABCA1 increased significantly in human myelin debris-treated HMC3 after cells were co-incubated with either K-604 (FIG. 9A) or F12511 (FIG. 9B) as the ACAT inhibitor for 24 hours. A similar effect was observed with N9 cells. In summary, the enhancing effects of myelin debris on cholesteryl ester biosynthesis were observed in mouse and human microglial cell lines. Further, the enhancing effects of ACAT1 inhibition on IL4R-α and ABCA1 protein levels in myelin debris-treated HMC3 cells and N9 cells was observed using two different ACAT inhibitors, K-604 and F12511.

### Example 6: Nanoparticle with or without F12511 Attenuates Neuroinflammation in ApoE3 and ApoE4 Mouse Brains

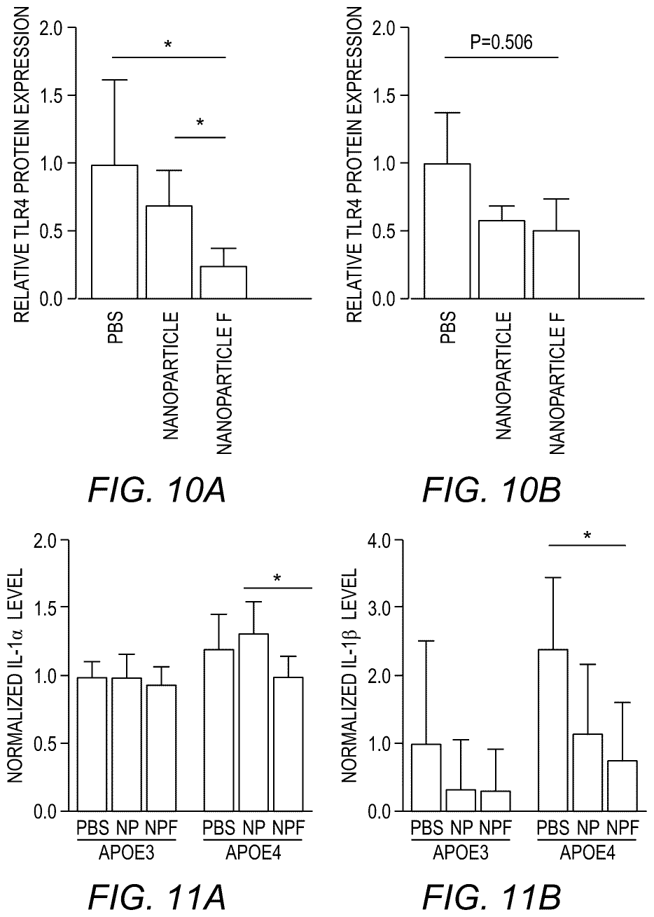

Genetic inactivation of Acat1 in the myeloid cell lineage suppresses lipopolysaccharide (LPS)-mediated neuroinflammation by modulating the fate of Toll-Like Receptor 4 (TLR4) in microglia. This indicates that inhibiting ACAT1 may produce an anti-inflammatory effect in the CNS by downregulating TLR4 protein expression. To demonstrate this, aging (16-20-month) female ApoE3 and ApoE4 knock-in (KI) mice were treated with two-week injections of PBS, or nanoparticle with or without F12511. Afterward, TLR4 protein expression in whole brain homogenates was determined. The results (FIGS. 10A and 10B) showed that, in both ApoE3 and ApoE4 female mouse forebrains, nanoparticle and F synergistically reduced TLR4 protein content.

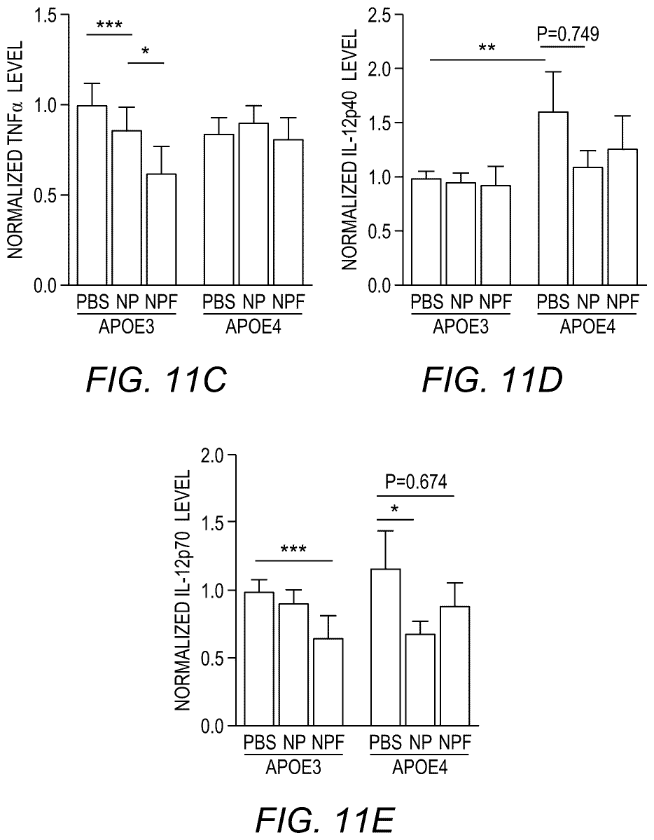

TLR4 is a major receptor at the plasma membrane and at the endosomal membrane that participate in pro-inflammatory signaling cascade. To further analyze expression of TLR4, aging female ApoE3/E4 KI mice were treated with nanoparticle with or without F12511 in the same manner as described above (i.e., 2-week IV), whole-brain homogenates were prepared, and Luminex technology was used to analyze the expression levels of 32 individual pro-inflammatory or anti-inflammatory cytokines (i.e., TNFa, Eotaxin, KC, MIP-1α, MIP-1B, RANTES, IL-15, GM-CSF, IL-7, MCP-1, IL-20p70, LIF, IL-3, IL-12p40, IL-5, IL-1β, IL-10, LIX, MIG, IP-10, M-CSF, VEGF, IL-13, IL-2, IL-9, IFNγ, and IL-1A). The results of the 5 individual pro-inflammatory cytokines were replotted (FIG. 11A-11E), and showed that nanoparticle alone and F12511 in nanoparticle synergistically suppress neuro-inflammation in both female aging ApoE3 and ApoE4 knock-in mice.

1: A method for reducing or attenuating neuroinflammation, amyloidopathy or tauopathy comprising administering to a subject in need thereof a pharmaceutical composition comprising nanoparticles comprising an outer lipid envelope and a core, the outer lipid envelope comprising distearoyl-sn-glycero-3-phosphoethanolamine-poly(ethylene glycol), the core comprising an Acyl-CoA:Cholesterol Acyltransferase (ACAT) inhibitor having the structure:

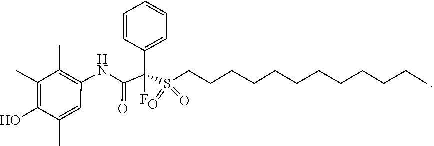 [SMILES: CCCCCCCCCCCCS(=O)(=O)[C@](F)(C(=O)Nc1cc(C)c(O)c(C)c1C)c1ccccc1]

2: The method of claim 1, wherein the ratio of ACAT inhibitor to phosphatidylcholine is in the range of about 0.3:1 to 2:1.

3: The method of claim 1, wherein said pharmaceutical composition is administered intravenously or intraperitoneally.

4: The method of claim 1, wherein the nanoparticle maintains a concentration of the ACAT inhibitor in the blood of the subject at above 20 μM for at least 4 hours.

5: The method of claim 1, further comprising administering a second therapeutic agent.

6: The method of claim 5, wherein the second therapeutic agent is a β-secretase inhibitor, γ-secretase modulator, proteasome inhibitor, small molecule activator of the unfolded protein response, Hsp90 inhibitor, small molecule Hsc70 inhibitor, deubiquitination enzyme inhibitor, epigallocatechin-3-gallate, variant of Hsp104 disaggregase, glutathione ethyl ester, or antibody.

7: The method of claim 1, wherein the outer lipid envelope further comprises phosphatidylcholine.

8: The method of claim 1, wherein ACAT activity in the brain of the subject is inhibited for at least 48 hours after administering the pharmaceutical composition to the subject.
